(19) United States
(12) Patent Application Publication
(10) Pub. No.: US 2015/0190079 A1
(43) Pub. Date: Jul. 9, 2015

(54) BIOLOGICAL SENSOR AND METHOD FOR MANUFACTURING BIOLOGICAL SENSOR

(71) Applicant: Murata Manufacturing Co., Ltd., Nagaokakyo-shi (JP)

(72) Inventors: Kazuhiro Yamaji, Nagaokakyo-shi (JP); Hirofumi Tsuchimoto, Nagaokakyo-shi (JP)

(21) Appl. No.: 14/666,747

(22) Filed: Mar. 24, 2015

Related U.S. Application Data
(63) Continuation of application No. PCT/JP2013/072057, filed on Aug. 19, 2013.

(30) Foreign Application Priority Data
Sep. 24, 2012 (JP) 2012-210019

(51) Int. Cl. A61B 5/1455; H05K 3/30; H05K 3/28; A61B 5/024

(52) U.S. Cl. CPC A61B 5/14551; A61B 5/02444; H05K 3/303; H05K 3/284; A61B 2562/12

(57) ABSTRACT

Provided is a biological sensor capable of reducing stray light that is received without passing through a biological body without increasing the size of the sensor, and capable of improving the reliability of the sensor.

A biological sensor includes a circuit board, a light-emitting element and a light-receiving element mounted on a main surface of the circuit board with a predetermined interval therebetween, a light-transmissive light-emitting element sealing portion formed only on an upper portion of a mounting region of the light-emitting element, a light-transmissive light-receiving element sealing portion formed only on an upper portion of a mounting region of the light-receiving element, and a light-blocking portion formed on the main surface of the circuit board and provided in respective peripheries of the light-emitting element sealing portion and the light-receiving element sealing portion and between the light-emitting element sealing portion and the light-receiving element sealing portion.

### CROSS REFERENCE TO RELATED APPLICATIONS

The present application is a continuation of PCT/JP2013/072057 filed Aug. 19, 2013, which claims priority to Japanese Patent Application No. 2012-210019, filed Sep. 24, 2012, the entire contents of each of which are incorporated herein by reference.

### FIELD OF THE INVENTION

The present invention relates to biological sensors that obtain biological signals and methods for manufacturing such biological sensors.

### BACKGROUND OF THE INVENTION

In recent years, people are taking an increased interest in managing, maintaining, and improving their health. As such, there is a desire for people to be able to more easily obtain biological information such as their pulse, electrocardiogram, and so on. Incidentally, conventional pulse monitors, pulse oximeters, and the like that obtain photoelectric pulse wave signals based on changes in the intensity of light that passes through a biological body such as a finger or is reflected by the biological body using a characteristic of bloodstream hemoglobin that absorbs visible light to infrared light are known (see Patent Documents 1, 2, and 3, for example).

Here, Patent Document 1 discloses a biological information measurement sensor provided with functions for both a biological body electrode and an oximeter probe. This biological information measurement sensor includes an electrode element mounted upon a high-polymer film, an LED serving as a light-emitting element and a PD serving as a light-receiving element affixed upon the electrode element with a predetermined interval therebetween, and AMPS serving as a conductive transparent gel that covers these elements. By providing such a configuration, when the sensor makes contact with the surface of the skin of a biological body, the electrode element makes contact with the skin via the conductive AMPS, and can therefore function as a normal electrode element. In addition, because the LED and PD make contact with the skin via the transparent AMPS, the oximeter probe function can be achieved as well.

Patent Document 2 discloses a photoelectric pulse sensor configured of a light-emitting element and a light-receiving element. This photoelectric pulse sensor includes a substrate to which the light-emitting element and the light-receiving element are attached and a base section configured of a light-transmissive material bonded to the substrate. A light-blocking plate that does not transmit light is inserted between a light-emitting portion (the light-emitting element) and a light-receiving portion (the light-receiving element) on the base section configured of the light-transmissive material.

Patent Document 3 discloses an optical sensor that detects pulsation of a blood flow as changes in light reflectance of a blood vessel, and detects a heartbeat based thereon. In this optical sensor, a light-emitting element is disposed in a central area of a casing when viewed from a light-transmissive surface; a light-blocking wall (partition wall) is formed in a shape surrounding the light-emitting element, and a plurality of light-receiving elements are disposed on an outer side portion of the light-blocking wall. An outer wall that surrounds the plurality of light-receiving elements on the outer side portion thereof and the light-blocking wall are connected by an intermediate wall. Meanwhile, individual spaces in the casing partitioned off by the light-blocking wall and the intermediate wall are filled with a light-transmissive resin.

Patent Document 1: Japanese Unexamined Utility Model Registration Application Publication No. 6-29504

Patent Document 2: Japanese Unexamined Utility Model Registration Application Publication No. 58-1402

Patent Document 3: Japanese Unexamined Utility Model Registration Application Publication No. 62-128505

As described above, in the biological information measurement sensor disclosed in Patent Document 1, the light-emitting element (LED) and the light-receiving element (PD) are covered by the conductive transparent gel (AMPS), and the LED and PD make contact with the skin of the biological body via the transparent AMPS. Accordingly, there is a risk that some of the light emitted from the LED (detection light) will reach the PD directly through the transparent AMPS when measurement is carried out. Normally, the intensity of light that is emitted from the LED and reaches the PD without passing through the biological body or without being reflected by the biological body (that is, stray light) is high in comparison with the intensity of light that has passed through the biological body or light reflected by the biological body. As such, there is a risk that the light originally to be detected, namely the light that has passed through the biological body or that has been reflected by the biological body, will be buried in the stray light (noise), leading to a drop in the signal to noise (S/N) ratio.

As opposed to this, in the photoelectric pulse sensor disclosed in Patent Document 2, the light-blocking plate that does not transmit light is inserted between the light-emitting portion and the light-receiving portion, and thus detection light that reaches the light-receiving element directly from the light-emitting element (stray light) can be blocked. However, in this photoelectric pulse sensor, there is a risk of ambient light (stray light) entering from a side surface of the base section configured of the light-transmissive material, resulting in the same risk of a drop in the signal to noise ratio as with the biological information measurement sensor disclosed in Patent Document 1.

In the optical sensor disclosed in Patent Document 3, as described above, the light-blocking wall is formed so as to surround the light-emitting element disposed in a central area of the casing and the plurality of light-receiving elements are disposed on the outer side portion of the light-blocking wall. Furthermore, the outer wall that surrounds the plurality of light-receiving elements on the outer side portion thereof and the light-blocking wall are connected by the intermediate wall. Thus according to this optical sensor, stray light that reaches the light-receiving element without passing through the biological body or without being reflected by the biological body can be blocked. However, this optical sensor also requires space for providing the stated light-blocking wall (partition wall), outer wall, and intermediate wall on the substrate, which increases the size of the sensor.

Furthermore, the light-transmissive resin used in the stated biological sensor generally has a high coefficient of linear expansion. The coefficient of linear expansion of the light-transmissive resin is approximately five times the coefficient of linear expansion of a typical glass epoxy substrate, for example. As such, the reliability of the sensor will drop when the light-transmissive resin, whose linear expansion coefficient is high, makes contact with the substrate on which the light-emitting element and the light-receiving element are mounted over a broad surface area. To be more specific, a difference in the coefficient of linear expansion between the light-transmissive resin and the substrate may cause the substrate to warp, the light-transmissive resin to separate from the substrate, or the like, for example. There is an additional risk of solder flush being produced at soldered areas of mounted electrical components during reflow and the like.

### SUMMARY OF THE INVENTION

Having been achieved to solve the aforementioned problems, it is an object of the present invention to provide a biological sensor that is capable of reducing stray light which does not pass through a biological body and is received without increasing the size of the sensor and that can offer increased reliability, as well as a method for manufacturing such a biological sensor.

A biological sensor according to the present invention includes: a circuit board; a light-emitting element and a light-receiving element mounted on a main surface of the circuit board with a predetermined interval therebetween; a light-transmissive light-emitting element sealing portion formed only on an upper portion of a mounting region of the light-emitting element; a light-transmissive light-receiving element sealing portion formed only on an upper portion of a mounting region of the light-receiving element; and a light-blocking portion formed on the main surface of the circuit board and provided in respective peripheries of the light-emitting element sealing portion and the light-receiving element sealing portion and between the light-emitting element sealing portion and the light-receiving element sealing portion.

According to the biological sensor of the present invention, the light-blocking portion is provided in the respective peripheries of the light-emitting element sealing portion and the light-receiving element sealing portion and between those portions. Accordingly, stray light incident on the light-receiving element without passing through the biological body is blocked by the light-blocking portion. Here, it is not necessary to provide a light-blocking wall or the like for blocking stray light on the circuit board, and thus stray light can be prevented without increasing the size of the circuit board (the biological sensor). In addition, according to the biological sensor of the present invention, the light-transmissive light-emitting element sealing portion is formed only on the upper portion of the mounting region of the light-emitting element, and the light-transmissive light-receiving element sealing portion is formed only on the upper portion of the mounting region of the light-receiving element. Accordingly, a surface area where a light-transmissive resin having a different coefficient of linear expansion makes contact with the circuit board can be reduced, which makes it possible to improve the reliability of the biological sensor. As a result, stray light that is received without passing through a biological body can be reduced without increasing the size of the sensor, and the reliability of the sensor can be improved.

In the biological sensor according to the present invention, it is preferable that the light-blocking portion, the light-emitting element sealing portion, and the light-receiving element sealing portion, which form a top surface of the biological sensor, be formed flush with each other.

By doing so, the surface of the biological sensor (the top surface) that makes contact with the finger or the like of a measurement subject is formed flat, and it is therefore possible to prevent imparting a sense of discomfort on the measurement subject when obtaining biological information such as a photoelectric pulse wave signal or the like, for example.

In the biological sensor according to the present invention, it is preferable that the light-emitting element sealing portion and the light-receiving element sealing portion project in a convex shape beyond a top surface of the light-blocking portion.

By doing so, the detection light emitted from the light-emitting element and the detection light incident on the light-receiving element can be focused, which makes it possible to improve the signal to noise ratio.

In the biological sensor according to the present invention, it is preferable that grooves be formed in the main surface of the circuit board, in the peripheries of the light-emitting element and the light-receiving element.

By doing so, when forming the light-emitting element sealing portion and the light-receiving element sealing portion, it is possible to prevent, for example, a pre-cure liquid-state light-transmissive resin from spreading over the grooves formed in the stated peripheries. Accordingly, the light-transmissive light-emitting element sealing portion and the light-receiving element sealing portion can be formed only on the upper portions of the mounting regions for the light-emitting element and the light-receiving element. Note that in this case, inner side portions of the grooves serve as the mounting regions of the light-emitting element and the light-receiving element.

In the biological sensor according to the present invention, it is preferable that the light-emitting element and the light-receiving element each be mounted on the circuit board via a sub-board.

By doing so, when forming the light-emitting element sealing portion and the light-receiving element sealing portion, it is possible to prevent, for example, a pre-cure liquid-state light-transmissive resin from spreading over the sub-boards. Accordingly, the light-transmissive light-emitting element sealing portion and the light-receiving element sealing portion can be formed only on the upper portions of the mounting regions for the light-emitting element and the light-receiving element. Note that in this case, mounting surfaces of the sub-boards serve as the mounting regions of the light-emitting element and the light-receiving element.

In the biological sensor according to the present invention, it is preferable that the light-emitting element and the light-receiving element each be surface-mounted type chip components. By doing so, the size of the biological sensor can be reduced.

In the biological sensor according to the present invention, it is preferable that the light-emitting element and the light-receiving element each be bare chip components. By doing so, mounting surface areas of the light-emitting element and the light-receiving element can be reduced, which makes it possible to further reduce the size of the biological sensor.

In the biological sensor according to the present invention, it is preferable that the light-emitting element sealing portion and the light-receiving element sealing portion each be formed of a resin that is light-transmissive with respect to a wavelength of detection light emitted by the light-emitting element.

By doing so, it is possible to cut ambient light (stray light) and allow only the detection light of a desired wavelength to be incident on the light-receiving element, which in turn makes it possible to further improve the signal to noise ratio.

In the biological sensor according to the present invention, it is preferable that the circuit board be formed in a rectangular shape, and the light-emitting element and the light-receiving element be mounted in corner areas on a diagonal line between opposing corners of the circuit board.

By doing so, the width of the circuit board can be reduced, and the size of the biological sensor (the circuit board) can be further reduced.

A method for manufacturing a biological sensor according to the present invention includes: a substrate formation process of forming a circuit board; a mounting process of mounting a light-emitting element and a light-receiving element on a main surface of the circuit board with a predetermined interval therebetween; a light-emitting element sealing process of forming a light-transmissive light-emitting element sealing portion only on an upper portion of a mounting region of the light-emitting element; a light-receiving element sealing process of forming a light-transmissive light-receiving element sealing portion only on an upper portion of a mounting region of the light-receiving element; and a forming process of forming a light-blocking portion on the main surface of the circuit board, in respective peripheries of the light-emitting element sealing portion and the light-receiving element sealing portion and between the light-emitting element sealing portion and the light-receiving element sealing portion.

According to the method for manufacturing a biological sensor of the present invention, the light-blocking portion is formed in the respective peripheries of the light-emitting element sealing portion and the light-receiving element sealing portion and between those portions. By doing so, a biological sensor capable of preventing stray light can be manufactured without forming a light-blocking wall or the like for blocking stray light on the circuit board, or in other words, without increasing the size of the sensor. In addition, according to the method for manufacturing a biological sensor of the present invention, the light-transmissive light-emitting element sealing portion is formed only on the upper portion of the mounting region of the light-emitting element, and the light-transmissive light-receiving element sealing portion is formed only on the upper portion of the mounting region of the light-receiving element. Accordingly, a surface area where a light-transmissive resin having a different coefficient of linear expansion makes contact with the circuit board can be reduced, which makes it possible to manufacture a highly-reliable biological sensor. As a result, stray light that is received without passing through a biological body can be reduced without increasing the size of the sensor, and a highly-reliable biological sensor can be manufactured.

It is preferable that the method for manufacturing a biological sensor according to the present invention further include a removal process of forming a top surface of the biological sensor configured of the light-blocking portion, the light-emitting element sealing portion, and the light-receiving element sealing portion so as to be flush with each other.

By doing so, the surface of the biological sensor (the top surface) that makes contact with the finger or the like of a measurement subject can be formed flat. Accordingly, it is possible to prevent imparting a sense of discomfort on the measurement subject when obtaining biological information such as a photoelectric pulse wave signal or the like, for example.

In the method for manufacturing a biological sensor according to the present invention, it is preferable that in the forming process, the light-blocking portion be formed so that apex portions of the light-emitting element sealing portion and the light-receiving element sealing portion each project in a convex shape beyond a top surface of the light-blocking portion.

In this case, the light-blocking portion is formed so that the respective apex portions of the light-emitting element sealing portion and the light-receiving element sealing portion project beyond the top surface of the light-blocking portion in a convex shape. Accordingly, the detection light emitted from the light-emitting element and the detection light incident on the light-receiving element can be focused, which makes it possible to manufacture a biological sensor having a high signal to noise ratio.

In the method for manufacturing a biological sensor according to the present invention, it is preferable that in the substrate formation process, grooves be formed in the main surface of the circuit board, in the respective peripheries of the light-emitting element and the light-receiving element.

By doing so, when forming the light-emitting element sealing portion and the light-receiving element sealing portion in the light-emitting element sealing process and the light-receiving element sealing process, it is possible to prevent, for example, the light-transmissive resin from spreading over the grooves formed in the stated peripheries. Accordingly, the light-transmissive light-emitting element sealing portion and the light-receiving element sealing portion can be formed only on the upper portions of the mounting regions for the light-emitting element and the light-receiving element.

In the method for manufacturing a biological sensor according to the present invention, it is preferable that in the mounting process, the light-emitting element and the light-receiving element each be mounted on the circuit board via a sub-board.

By doing so, when forming the light-emitting element sealing portion and the light-receiving element sealing portion in the light-emitting element sealing process and the light-receiving element sealing process, it is possible to prevent, for example, the light-transmissive resin from spreading over the sub-board. Accordingly, the light-transmissive light-emitting element sealing portion and the light-receiving element sealing portion can be formed only on the upper portions of the mounting regions for the light-emitting element and the light-receiving element.

According to the present invention, stray light that is received without passing through a biological body can be reduced without increasing the size of a sensor, and the reliability of the sensor can be improved.

### BRIEF DESCRIPTION OF THE DRAWINGS

FIG. 1 is a vertical cross-sectional view of a biological sensor according to an embodiment.

FIG. 2 is a plan view illustrating the arrangement, on a circuit board, of a light-emitting element and a light-receiving element that partially configure a biological sensor according to an embodiment.

FIG. 3 is a diagram illustrating a process for manufacturing (a method for manufacturing) a biological sensor according to an embodiment.

FIG. 4 is a vertical cross-sectional view of a biological sensor according to a first variation.

FIG. 5 is a vertical cross-sectional view of a biological sensor according to a second variation.

FIG. 6 is a vertical cross-sectional view of a biological sensor according to a third variation.

FIG. 7 is a vertical cross-sectional view of a biological sensor according to a fourth variation.

### DETAILED DESCRIPTION OF THE PREFERRED EMBODIMENTS

Hereinafter, preferred embodiments of the present invention will be described in detail with reference to the drawings. Note that the same reference numerals are appended to identical elements and redundant descriptions thereof will be omitted.

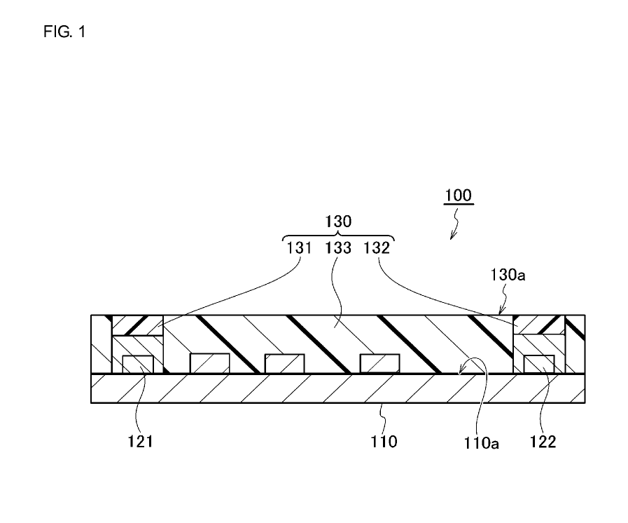

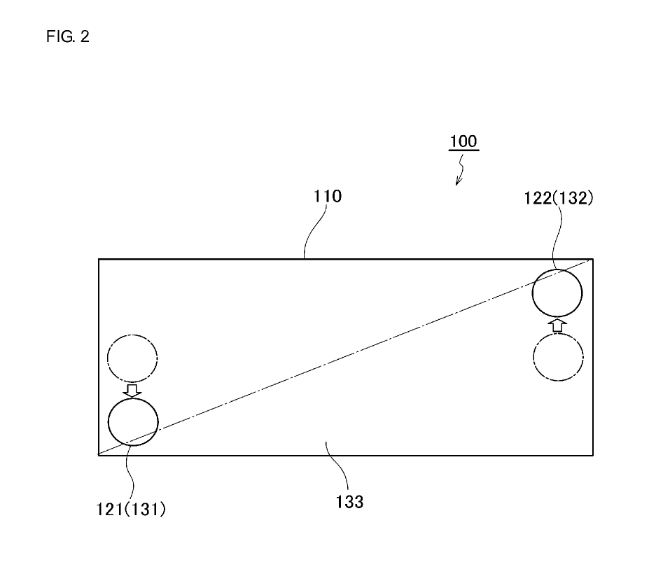

First, the configuration of a biological sensor 100 according to an embodiment will be described using FIGS. 1 and 2. FIG. 1 is a vertical cross-sectional view of the biological sensor 100. FIG. 2 is a plan view illustrating the arrangement, on a circuit board 110, of a light-emitting element 121 and a light-receiving element 122 that partially configure the biological sensor 100 according to an embodiment.

The biological sensor 100 is a sensor that detects (measures) biological information such as a pulse, an oxygen saturation level, or the like, for example, by making contact with a fingertip or the like. The biological sensor 100 optically measures the pulse, the oxygen saturation level, or the like using a characteristic of bloodstream hemoglobin that absorbs light.

Accordingly, the biological sensor 100 is configured including the circuit board 110, the light-emitting element 121 and the light-receiving element 122 mounted on a main surface 110a of the circuit board 110, and a sealing portion 130 formed on the main surface 110a of the circuit board 110. Note that the sealing portion 130 is configured including a light-emitting element sealing portion 131, a light-receiving element sealing portion 132, and a light-blocking portion 133.

The circuit board 110 is a thin, plate-shaped substrate formed in a horizontally long rectangular shape from an insulative material (a dielectric) such as an insulative resin, a ceramic material, or the like, for example. The light-emitting element 121, the light-receiving element 122, and various types of electronic components are mounted on the main surface (mounting surface) 110a of the circuit board 110. Here, it is preferable that the light-emitting element 121 and the light-receiving element 122 be mounted in corner positions on a diagonal line between opposing corners of the circuit board 110, as illustrated in FIG. 2. The light-emitting element 121 and the light-receiving element 122 are mounted with a predetermined interval, such as an interval of approximately 4-20 mm, provided therebetween.

The light-emitting element 121 emits, for example, near-infrared light whose absorption coefficient in hemoglobin is high. On the other hand, the light-receiving element 122 receives light that has been emitted from the light-emitting element 121 and has passed through a biological body or been reflected by a biological body (detection light) and outputs an electrical signal based on the intensity of the received light.

An LED, a VCSEL (Vertical Cavity Surface Emitting LASER), a resonator-type LED, or the like can be used as the light-emitting element 121. A photodiode (PD), a phototransistor, or the like can be preferably used as the light-receiving element 122. Note that a surface-mounted type (SMD: Surface Mount Device) chip component (package component) can be used preferably as the light-emitting element 121 and the light-receiving element 122.

The sealing portion 130 is formed in a parallelepiped shape on the main surface 110a of the circuit board 110, and is configured of the light-emitting element sealing portion 131 that seals the light-emitting element 121, the light-receiving element sealing portion 132 that seals the light-receiving element 122, and the light-blocking portion 133.

The light-emitting element sealing portion 131 is formed of a light-transmissive resin in, for example, a cylindrical shape (or a circular truncated cone shape), and seals the light-emitting element 121. The light-emitting element sealing portion 131 is formed only on an upper area of a component top surface (corresponding to a “mounting region” in the claims) of the light-emitting element 121. A transparent epoxy resin or the like, for example, is used as the light-transmissive resin that forms the light-emitting element sealing portion 131. Here, to ensure that light aside from the detection light, which has a desired wavelength, is absorbed and cut, it is preferable that the light-emitting element sealing portion 131 be formed of a resin that is light-transmissive with respect to the wavelength of the detection light (infrared light, for example) emitted from the light-emitting element 121.

Meanwhile, the light-receiving element sealing portion 132 is formed of a light-transmissive resin in, for example, a cylindrical shape (or a circular truncated cone shape), and seals the light-receiving element 122. The light-receiving element sealing portion 132 is formed only on an upper area of a component top surface (corresponding to a “mounting region” in the claims) of the light-receiving element 122. A transparent epoxy resin or the like, for example, is used as the light-transmissive resin that forms the light-receiving element sealing portion 132. Here, to ensure that light aside from the detection light, which has a desired wavelength, is absorbed and cut, it is preferable that the light-receiving element sealing portion 132 be formed of a resin that is light-transmissive with respect to the wavelength of the detection light (infrared light, for example) emitted from the light-emitting element 121.

The light-blocking portion 133 is formed by filling regions that surround the light-emitting element sealing portion 131 and the light-receiving element sealing portion 132, respectively, and a region between the light-emitting element sealing portion 131 and the light-receiving element sealing portion 132, on the main surface 110a of the circuit board 110 with a light-blocking resin. The light-blocking portion 133 defines four side surfaces of the sealing portion 130. Note that an epoxy resin or the like that contains a light-blocking powder such as carbon black is used preferably as the light-blocking portion 133.

Surfaces of upper portions of the light-emitting element sealing portion 131, the light-receiving element sealing portion 132, and the light-blocking portion 133, respectively, define a top surface 130a of the sealing portion 130. In the present embodiment, apex portions of the light-emitting element sealing portion 131, the light-receiving element sealing portion 132, and the light-blocking portion 133, respectively, that form the top surface 130a of the biological sensor 100, are formed so as to be flush with each other. In other words, the top surface 130a of the sealing portion 130 is formed flat.

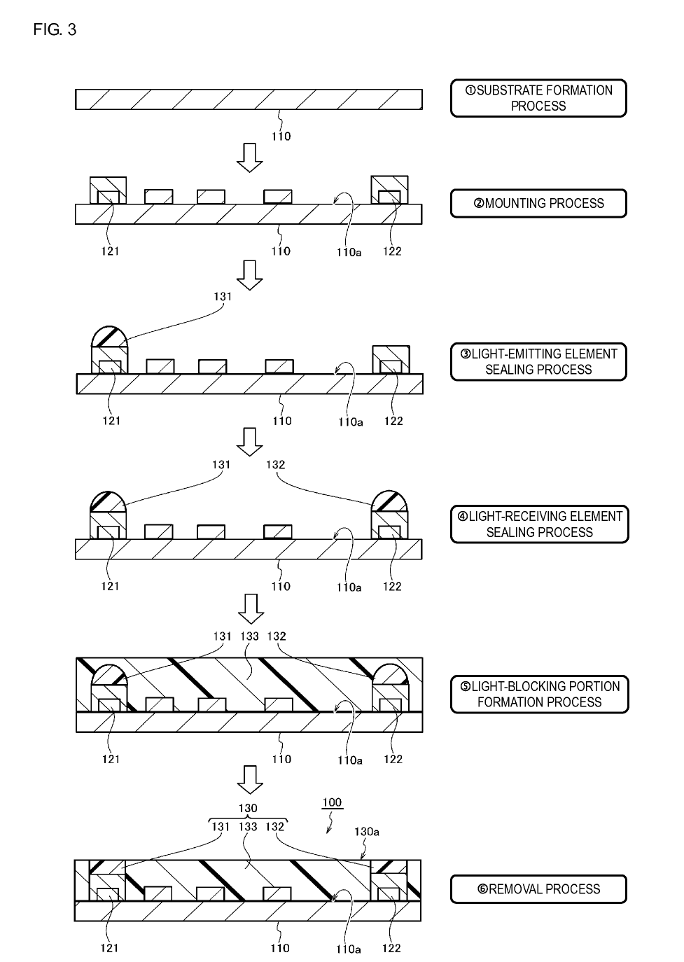

Next, a process for manufacturing (a method for manufacturing) the biological sensor 100 according to the present embodiment will be described with reference to FIG. 3. Here, FIG. 3 is a diagram illustrating a process for manufacturing (a method for manufacturing) the biological sensor 100. As illustrated in FIG. 3, the process for manufacturing the biological sensor 100 primarily includes a substrate formation process, a mounting process, a light-emitting element sealing process, a light-receiving element sealing process, a light-blocking portion formation process, and a removal process. These processes will be described hereinafter.

(Substrate Formation Process)

First, in the substrate formation process, the thin plate-shaped circuit board 110 in which a wiring pattern is formed through etching or the like is formed in a horizontally long rectangular shape from an insulative resin, a ceramic material, or the like, for example.

(Mounting Process)

Next, in the mounting process, the light-emitting element 121, the light-receiving element 122, and various types of electrical components are soldered and mounted in predetermined positions on the main surface (mounting surface) 110a of the circuit board 110.

(Light-Emitting Element Sealing Process)

In the light-emitting element sealing process, a light-transmissive resin is spread, for example (or molded, or affixed in a solid state) onto the component top surface of the light-emitting element 121. Through this, the light-transmissive light-emitting element sealing portion 131 is formed only on an upper portion of the component top surface (the mounting region) of the light-emitting element 121. Note that here, the light-emitting element sealing portion 131 is formed having a bell shape (or an inverse U-shape), for example.

(Light-Receiving Element Sealing Process)

Meanwhile, in the light-receiving element sealing process, a light-transmissive resin is spread, for example (or molded, or affixed in a solid state) onto the component top surface of the light-receiving element 122, in the same manner as in the aforementioned light-emitting element sealing process. Through this, the light-transmissive light-receiving element sealing portion 132 is formed only on an upper portion of the component top surface (the mounting region) of the light-receiving element 122. Note that here, the light-receiving element sealing portion 132 is formed having a bell shape (or an inverse U-shape), for example. The light-emitting element sealing process and the light-receiving element sealing process may be carried out simultaneously.

(Light-Blocking Portion Formation Process)

Next, in the light-blocking portion formation process, the light-blocking portion 133 is formed by filling regions that surround the light-emitting element sealing portion 131 and the light-receiving element sealing portion 132, respectively, and a region between the light-emitting element sealing portion 131 and the light-receiving element sealing portion 132, on the main surface 110a of the circuit board 110 with an epoxy resin or the like that contains a light-blocking powder such as carbon black. Note that here, the light-blocking resin is filled to a height that is higher than the light-emitting element sealing portion 131 and the light-receiving element sealing portion 132, or in other words, so that the light-emitting element sealing portion 131 and the light-receiving element sealing portion 132 are completely encapsulated.

(Removal Process)

In the removal process, the apex portions of the light-emitting element sealing portion 131, the light-receiving element sealing portion 132, and the light-blocking portion 133 are formed flush with each other by being ground down through polishing or the like, for example. As a result, the apex portions of the light-emitting element sealing portion 131 and the light-receiving element sealing portion 132 are exposed at the top surface 130a of the biological sensor 100 (the sealing portion 130), and the top surface 130a of the biological sensor 100 (the sealing portion 130) is formed flat. Note that in this process, the upper portions of the bell-shaped (or inverse U-shaped) light-emitting element sealing portion 131 and light-receiving element sealing portion 132 formed in the light-emitting element sealing process and the light-receiving element sealing process are cut away, and the light-emitting element sealing portion 131 and the light-receiving element sealing portion 132 are formed in a cylindrical shape (or a circular truncated cone shape). The biological sensor 100 is manufactured as described thus far.

According to the biological sensor 100 of the present embodiment, a biological signal is detected by bringing the surface (the top surface 130a) of the biological sensor 100 into contact with the fingertip of a measurement subject, for example. In the case where the surface of the biological sensor 100 has been brought into contact with the fingertip, the light emitted from the light-emitting element 121 passes through the light-emitting element sealing portion 131 and is incident on the fingertip. The light that is incident on the fingertip and passes through the fingertip then enters an opening portion of the light-receiving element sealing portion 132. The light then passes through the light-receiving element sealing portion 132 and is received by the light-receiving element 122. A change in the intensity of the detection light that has passed through the fingertip is obtained as a photoelectric pulse wave signal.

As described thus far, according to the biological sensor 100 of the present embodiment, the light-blocking portion 133 is provided in the respective peripheries of the light-emitting element sealing portion 131 and the light-receiving element sealing portion 132 and between those portions. Accordingly, stray light incident on the light-receiving element 122 without passing through the biological body is blocked by the light-blocking portion 133. Here, according to the biological sensor 100, it is not necessary to provide a light-blocking wall or the like for blocking stray light on the circuit board 110, and thus stray light can be prevented (that is, the signal to noise ratio can be improved) without increasing the size of the circuit board 110 (the biological sensor 100). Furthermore, according to the biological sensor 100, the light-transmissive light-emitting element sealing portion 131 is formed only on the upper portion of the component top surface (the mounting region) of the light-emitting element 121, and the light-transmissive light-receiving element sealing portion 132 is formed only on the upper portion of the component top surface (the mounting region) of the light-receiving element 122. Accordingly, a surface area where a light-transmissive resin having a different coefficient of linear expansion makes contact with the circuit board 110 can be reduced, which makes it possible to improve the reliability of the biological sensor 100. As a result, according to the biological sensor 100, stray light that is received without passing through a biological body can be reduced without increasing the size of the sensor, and the reliability of the sensor can be improved.

In addition, according to the biological sensor 100 of the present embodiment, the surface of the biological sensor 100 (the top surface 130a of the sealing portion 130) that makes contact with the finger or the like of the measurement subject is formed flat, and it is therefore possible to prevent imparting a sense of discomfort on the measurement subject when obtaining a biological signal such as a photoelectric pulse wave signal or the like, for example.

According to the biological sensor 100 of the present embodiment, the light-emitting element sealing portion 131 and the light-receiving element sealing portion 132 are each formed of a resin that is light-transmissive with respect to the wavelength of the detection light emitted by the light-emitting element 121, or in other words, that selectively allows only the detection light having a desired wavelength to pass therethrough, which makes it possible to cut ambient light (stray light) and allow only the detection light to be incident on the light-receiving element 122. This makes it possible to improve the signal to noise ratio.

Furthermore, according to the biological sensor 100 of the present embodiment, the circuit board 110 is formed in a rectangular shape, and the light-emitting element 121 and the light-receiving element 122 are mounted in corner areas on a diagonal line between opposing corners of the circuit board 110; this makes it possible to reduce the width of the circuit board 110 and further reduce the size of the biological sensor 100 (the circuit board 110).

(First Variation)

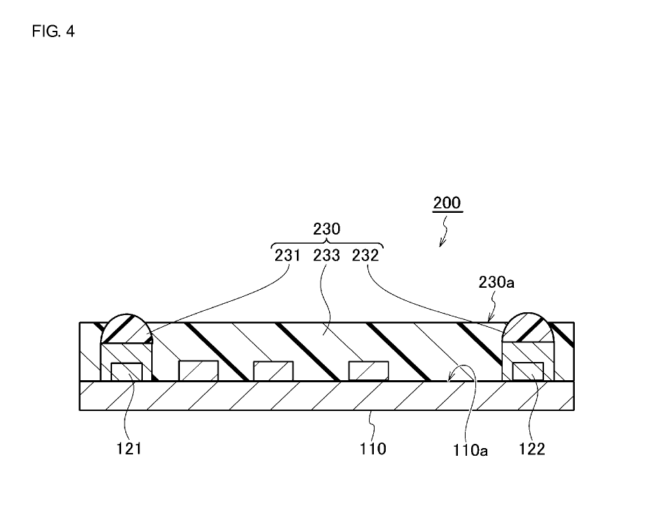

Although the surface of the biological sensor 100 (the top surface 130a of the sealing portion 130) is formed flat, or in other words, the apex portions of the light-emitting element sealing portion 131, the light-receiving element sealing portion 132, and the light-blocking portion 133 are each formed so as to be flush with each other in the aforementioned embodiment, apex portions of a light-emitting element sealing portion 231 and a light-receiving element sealing portion 232 may be formed so as to project in a convex shape from the top surface of a light-blocking portion 233, as illustrated in FIG. 4. Here, FIG. 4 is a vertical cross-sectional view of a biological sensor 200 according to a first variation.

The light-emitting element sealing portion 231 and the light-receiving element sealing portion 232 according to the present variation are each formed having a bell shape (or an inverse U shape), for example, and as described above, the apex portions thereof are formed so as to project in a convex shape (a lens shape) from the top surface of the light-blocking portion 233. The other configurations are the same or similar to those in the aforementioned biological sensor 100, and thus detailed descriptions thereof will be omitted.

When manufacturing the biological sensor 200 according to the present variation, the light-blocking portion 233 is, in the aforementioned formation processes, formed so that the respective apex portions of the light-emitting element sealing portion 231 and the light-receiving element sealing portion 232 project from a top surface 230a of a sealing portion 230. In other words, a light-blocking resin is filled to a height that is lower than the light-emitting element sealing portion 231 and the light-receiving element sealing portion 232. In addition, in this case, the aforementioned removal process is unnecessary.

According to the biological sensor 200 of the first variation, the detection light emitted from the light-emitting element 121 and the detection light incident on the light-receiving element 122 can be focused, which makes it possible to further improve the signal to noise ratio.

(Second Variation)

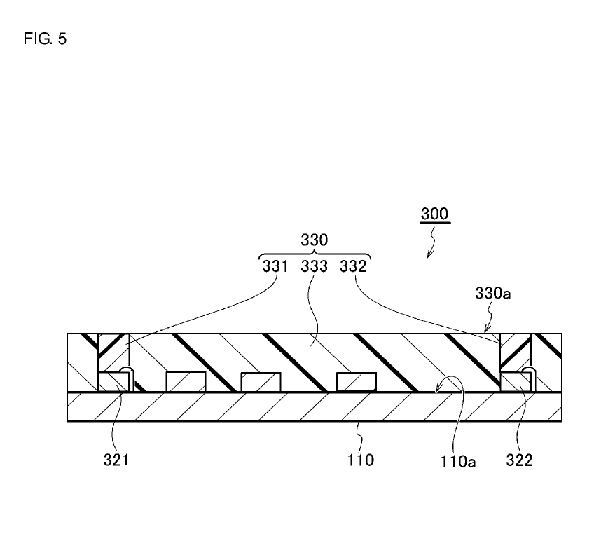

Although surface-mounted type (SMD) chip components are used for the light-emitting element 121 and the light-receiving element 122 in the aforementioned embodiment, it is also preferable that bare chip components 321 and 322 be used, as illustrated in FIG. 5. Here, FIG. 5 is a vertical cross-sectional view of a biological sensor 300 according to a second variation. The other configurations are the same or similar to those in the aforementioned biological sensor 100, and thus detailed descriptions thereof will be omitted.

According to the biological sensor 300 of the present variation, mounting surface areas of a light-emitting element 321 and a light-receiving element 322 can be reduced, which makes it possible to further reduce the size of the biological sensor 300.

(Third Variation)

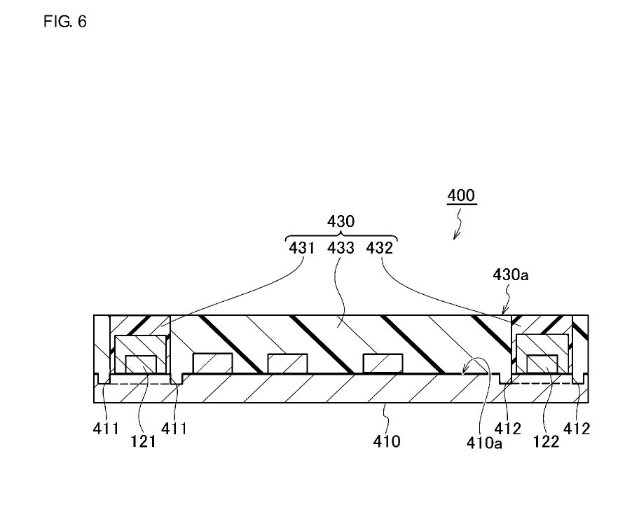

Next, the configuration of a biological sensor 400 according to a third variation will be described with reference to FIG. 6. Here, FIG. 6 is a vertical cross-sectional view of the biological sensor 400 according to the third variation. The biological sensor 400 differs from the aforementioned biological sensor 100 in that a circuit board 410, in which grooves 411 and 412 are formed in the respective peripheries of the light-emitting element 121 and the light-receiving element 122, is used instead of the aforementioned circuit board 110. The other configurations are the same or similar to those in the aforementioned biological sensor 100, and thus detailed descriptions thereof will be omitted.

When manufacturing the biological sensor 400 according to the present variation, the grooves 411 and 412 are formed in a main surface 410a of the circuit board 410, in the respective peripheries of the light-emitting element 121 and the light-receiving element 122, during the aforementioned substrate formation process, through a cutting process or the like, for example.

According to the biological sensor 400 of the present variation, when forming a light-emitting element sealing portion 431 and a light-receiving element sealing portion 432, it is possible to prevent, for example, a pre-cure liquid-state light-transmissive resin from spreading over the grooves 411 and 412 formed in the stated peripheries. Accordingly, the light-transmissive light-emitting element sealing portion 431 and light-receiving element sealing portion 432 can be formed only on the upper portions of the mounting regions for the light-emitting element 121 and the light-receiving element 122. This method is particularly useful when using bare chip components that do not have cases as the light-emitting element 121 and the light-receiving element 122. Meanwhile, in this case, inner side portions of the grooves 411 and 412 serve as the mounting regions (the mounting region denoted in the claims) of a light-emitting element 431 and a light-receiving element 432.

(Fourth Variation)

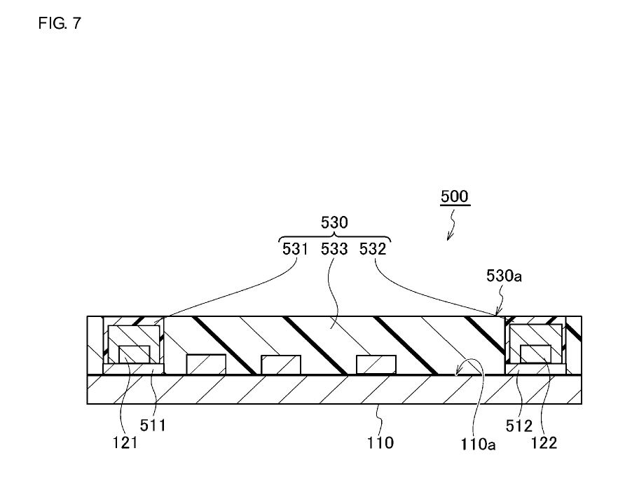

Next, the configuration of a biological sensor 500 according to a fourth variation will be described with reference to FIG. 7. Here, FIG. 7 is a vertical cross-sectional view of the biological sensor 500 according to the fourth variation. The biological sensor 500 differs from the aforementioned biological sensor 100 in that the light-emitting element 121 and the light-receiving element 122 are mounted on the circuit board 110 via sub-boards 511 and 512 (or spacers), respectively. The other configurations are the same or similar to those in the aforementioned biological sensor 100, and thus detailed descriptions thereof will be omitted.

When manufacturing the biological sensor 500 according to the present variation, first, in the aforementioned mounting process, the light-emitting element 121 and the light-receiving element 122 are mounted to the sub-boards 511 and 512 through soldering. Then, the sub-boards 511 and 512 on which the light-emitting element 121 and the light-receiving element 122 are soldered to the circuit board 110.

According to the biological sensor 500 of the present variation, when forming a light-emitting element sealing portion 531 and a light-receiving element sealing portion 532, it is possible to prevent, for example, a pre-cure liquid-state light-transmissive resin from spreading over the sub-boards 511 and 512. Accordingly, the light-transmissive light-emitting element sealing portion 531 and light-receiving element sealing portion 532 can be formed only on the upper portions of the mounting regions for the light-emitting element 121 and the light-receiving element 122. This method is particularly useful when using bare chip components that do not have cases as the light-emitting element 121 and the light-receiving element 122. Meanwhile, in this case, mounting surfaces of the aforementioned sub-boards 511 and 512 serve as the mounting regions (the mounting region denoted in the claims) of the light-emitting element 121 and the light-receiving element 122.

Although embodiments of the present invention have been described thus far, the present invention is not intended to be limited to the aforementioned embodiments, and many variations can be carried out thereon. For example, although a single light-emitting element 121 is provided in the aforementioned embodiments, a plurality of light-emitting elements may be provided. More specifically, two light-emitting elements that emit different wavelengths of light in order to obtain an abundance ratio between oxygenated hemoglobin and reduced hemoglobin, which indicates the blood oxygen saturation level, may be provided. In this case, it is preferable that one of the light-emitting elements emit near-infrared light whose absorption coefficient in oxygenated hemoglobin is high, and that the other light-emitting element emit near-infrared light whose absorption coefficient in reduced hemoglobin is high. Furthermore, in this case, a light-emitting element sealing portion is formed on the upper portion of each light-emitting element.

In addition, the shapes of the light-emitting element sealing portion 131 and the light-receiving element sealing portion 132 are not limited to those described in the aforementioned embodiment (a cylindrical shape or a circular truncated cone shape). The light-emitting element sealing portion 131 and the light-receiving element sealing portion 132 may be formed having a square column shape, a truncated square pyramid shape, or the like, for example, in accordance with the shapes of the light-emitting element and light-receiving element (package shapes).

### REFERENCE SIGNS LIST

100, 200, 300, 400, 500 biological sensor
110, 410 circuit board
121, 321 light-emitting element
122, 322 light-receiving element
130, 230, 330, 430, 530 sealing portion
131, 231, 331, 431, 531 light-emitting element sealing portion
132, 232, 332, 432, 532 light-receiving element sealing portion
133, 233, 333, 433, 533 light-blocking portion
411, 412 groove
511, 512 sub-board

1. A biological sensor comprising:
a circuit board;
a light-emitting element disposed on a surface of the circuit board;
a light-receiving element disposed on the surface of the circuit board with a predetermined distance between the light-emitting element and the light-receiving element;
a first light-transmissive sealing member disposed on a mounting region of the light-emitting element;
a second light-transmissive sealing member disposed on a mounting region of the light-receiving element; and
a light-blocking element disposed on the surface of the circuit board between the first and second light-transmissive sealing members.

2. The biological sensor according to claim 1, wherein the light-blocking element is disposed in respective peripheries of the first and second light-transmissive sealing members.

3. The biological sensor according to claim 1, wherein the first and second light-transmissive sealing members are disposed only on the mounting regions of the light-emitting element and the light-receiving element, respectively.

4. The biological sensor according to claim 1, wherein the light-blocking element and the first and second light-transmissive sealing members collectively form a top surface of the biological sensor and are flush with each other.

5. The biological sensor according to claim 1, wherein the first and second light-emitting sealing members project from the surface of the circuit board in a convex shape beyond the light-blocking element.

6. The biological sensor according to claim 1, wherein the surface of the circuit board comprises grooves disposed in peripheries of the light-emitting element and the light-receiving element.

7. The biological sensor according to claim 6, further comprising a pair of sub-boards disposed between respective grooves of the surface of the circuit board wherein the light-emitting element and the light-receiving element are mounted on the circuit board via the pair of sub-boards, respectively.

8. The biological sensor according to claim 1, wherein the light-emitting element and the light-receiving element are surface-mounted chips.

9. The biological sensor according to claim 1, wherein the light-emitting element and the light-receiving element are bare chip components.

10. The biological sensor according to claim 1, wherein the first and second light-emitting sealing members comprise a resin that is light-transmissive with respect to a wavelength of light emitted by the light-emitting element.

11. The biological sensor according to claim 1, wherein the circuit board comprises a rectangular shape and the light-emitting element and the light-receiving element are mounted in corner areas of the circuit board on a diagonal line between opposing corners of the circuit board.

12. A method for manufacturing a biological sensor comprising:
mounting a light-emitting element on a surface of a circuit board;
mounting a light-receiving element on the surface of the circuit board with a predetermined distance between the light-emitting element and the light-receiving element;
forming a first light-transmissive sealing member on a mounting region of the light-emitting element;
forming a second light-transmissive sealing member on a mounting region of the light-receiving element; and
forming a light-blocking element on the surface of the circuit board between the light-emitting element sealing portion and the light-receiving element sealing portion.

13. The method for manufacturing a biological sensor according to claim 12, wherein the forming of the light-blocking element comprises forming the light-blocking element in respective peripheries of the first and second light-transmissive sealing members.

14. The method for manufacturing a biological sensor according to claim 12, wherein the forming of the first and second light-transmissive sealing members comprises forming the first and second light-transmissive sealing members only on the mounting regions of the light-emitting element and the light-receiving element, respectively.

15. The method for manufacturing a biological sensor according to claim 12, further comprising:
removing a portion of each of the light-blocking element, the first light-transmissive sealing member, and the second light-transmissive sealing member to form a top surface of the biological sensor with the light-blocking element and the first and light-transmissive sealing members flush with each other.

16. The method for manufacturing a biological sensor according to claim 12, wherein the forming of the light-blocking element comprises forming the light-blocking element so that respective apex portions of the first and light-transmissive sealing members each project in a convex shape beyond a top surface of the light-blocking portion.

17. The method for manufacturing a biological sensor according to claim 12, further comprising forming grooves in the surface of the circuit board before mounting the light-emitting element and the light-receiving element on the surface of the circuit board.

18. The method for manufacturing a biological sensor according to claim 17, wherein the grooves are form in respective peripheries of the light-emitting element and the light-receiving element.

19. The method for manufacturing a biological sensor according to claim 12, further comprising mounting a pair of sub-boards to the surface of the circuit board and mounting the light-emitting element and the light-receiving element to the pair of sub-boards, respectively.

20. The method for manufacturing a biological sensor according to claim 12, wherein the forming of the light-blocking element comprises filling regions between the light-emitting element sealing portion and the light-receiving element sealing portion with an epoxy resin having a light-blocking powder.
